(19) United States
(12) Patent Application Publication
(10) Pub. No.: US 2013/0345310 A1
(43) Pub. Date: Dec. 26, 2013

(54) SOLID COMPOSITION CONTAINING THE INGREDIENT RASAGILINE

(71) Applicants: Katrin Rimkus, Iserlohn (DE); Sandra Brueck, Ottenhofen (DE); Frank Muskulus, Laupheim (DE); Jana Paetz, Bonn (DE)

(72) Inventors: Katrin Rimkus, Iserlohn (DE); Sandra Brueck, Ottenhofen (DE); Frank Muskulus, Laupheim (DE); Jana Paetz, Bonn (DE)

(73) Assignee: RATIOPHARM GMBH, Ulm (DE)

(21) Appl. No.: 13/967,240

(22) Filed: Aug. 14, 2013

Related U.S. Application Data
(63) Continuation of application No. 13/140,402, filed on Sep. 6, 2011.

(30) Foreign Application Priority Data
Dec. 19, 2008 (DE) 102008064061.1

(51) Int. Cl. A61K 31/135

(52) U.S. Cl. CPC A61K 31/135
USPC 514/657

(57) ABSTRACT

The present invention relates to a solid composition containing at least one pharmaceutically compatible auxiliary ingredient and rasagiline or a pharmaceutically compatible salt thereof as an active ingredient. The invention further relates to a method for producing said solid composition and to a medicine comprising said solid composition.

The present invention relates to a solid composition containing at least one pharmaceutically acceptable excipient and rasagiline or a pharmaceutically acceptable salt thereof as the active ingredient. The invention also relates to a method for producing this solid composition as well as a pharmaceutical drug containing this solid composition.

Rasagiline, the R(+)-enantiomer of N-propargyl-1-aminoindan, is a known active ingredient, which is used for treatment of Parkinson's disease, dementia and Alzheimer's disease in particular. For example, U.S. Pat. No. 5,532,415 discloses the production of rasagiline and various salts of this compound as well as the use of the active ingredient for treatment of a number of diseases such as Parkinson's disease, memory disorders, dementia, depression, schizophrenia, hyperactivity, etc. Another method for producing salts of rasagiline is disclosed in WO 2002/068376.

Rasagiline is described as a single compound in EP-A-0 436 492 which discloses rasagiline and pharmaceutically acceptable acid addition salts thereof in general, specifically the hydrochloride and the tartrate of rasagiline. Additional salts of rasagiline are described in WO 95/11016, namely the sulfate, phosphate, mesylate, maleate, esylate, acetate, fumarate, hydrobromide, tosylate and benzoate. Additional acid addition salts of rasagiline are described in WO 2008/019871.

Active ingredients often in finely ground form are mixed as a powder with various excipients in pharmaceutical formulations and pressed to form tablets, optionally after granulation. For this purpose it is important first that the physical form of the active ingredient, i.e., the polymorphic or amorphous form, does not change during storage of the drug. Secondly, it is important that the active ingredient can be mixed well with the excipients that are used and can be processed further. In particular when working with small quantities of active ingredient, a uniform distribution within the quantities of excipient used is necessary to ensure that, for example, tablets produced from the mixture always contain the same quantities of active ingredient.

WO 2008/131961 is concerned with the problem of the stability of the physical form of the active ingredient rasagiline used this patent discloses adsorbates which contain a pharmaceutically acceptable salt of rasagiline in amorphous form in addition to a pharmaceutically acceptable water-soluble organic active ingredient. The adsorbates are prepared by spray drying a solution of the active ingredient and excipient. In this way the active ingredient is adsorbed onto the excipient particles so that there are separate phases of the active ingredient and the excipient.

WO 2006/091657 is concerned with the problem of homogeneous distribution of active ingredient particles in excipient mixtures. An especially uniform distribution of the active ingredient particles in an excipient mixture is achieved by milling the pharmaceutically acceptable salt of rasagiline in such a way that more than 90% by volume of the active ingredient particles is less than 250 μm in size. Active ingredient particles micronized in this way, however, have the disadvantage that electrostatic charge buildup and formation of agglomerates may occur in milling and further processing. Furthermore, micronized active ingredients have a reduced flowability, which may result in problems in further processing.

There is therefore still a need for forms of the active ingredient rasagiline which can be prepared easily and processed further in such a way that a uniform distribution in pharmaceutical formulations can be produced easily and reliably. This is especially important with low-dose active ingredients such as rasagiline.

One object of the present invention is thus to make available the active ingredient rasagiline in a form which does not have the disadvantages of the state of the art. In particular it should be easy to process this form, optionally with additional excipients, to produce the pharmaceutical drugs and thereby ensure a uniform distribution of the active ingredient and thus uniformity of the active ingredient content in the pharmaceutical drugs. Furthermore, the active ingredient form in the finished pharmaceutical drugs should be physically stable during storage in particular.

It has now been found that the active ingredient rasagiline and its pharmaceutically acceptable salts can be processed with a pharmaceutically acceptable excipient to form a solid composition in which the excipient and the active ingredient are present in a homogeneous mixture containing the active ingredient in a noncrystalline form. Instead the active ingredient and excipient are present here in the form of a solid solution in which they are mixed at a molecular level.

The present invention thus relates to a solid composition containing at least one pharmaceutically acceptable excipient and rasagiline or a pharmaceutically acceptable salt thereof as the active ingredient, characterized in that the excipient and the active ingredient are present in a homogeneous, molecularly disperse mixture.

A homogeneous mixture in the present case is understood to be a solid solution of the active ingredient in the excipient. The solid composition thus has only one phase, with the dissolved active ingredient being present in a uniform distribution in the excipient. The homogeneous mixture thus contains at least largely no phases of pure excipient or pure active ingredient. The excipient and active ingredient are instead mixed together on a molecular level so that no phase boundaries between the excipient and active ingredient can be observed either visually or with other physical methods. Accordingly, the active ingredient is not present in either crystalline or amorphous form in the solid composition according to the invention. The active ingredient is instead distributed between the molecules of the excipient on a molecular level. Therefore the active ingredient can no longer be detected by, for example, x-ray powder diffractograms or by spectroscopic methods such as confocal Raman spectroscopy in which pressed tablets of the samples are prepared so that confocal mapping can be performed on the smooth surface of these tablets. This mapping is performed in an area of 10 μm×10 μm. For these measurements a Raman spectrometer from the company NT-MDT (NTEGRA Spektra Nanofinder) with a maximal lateral resolution of Δx, Δy<400 nm, vertical resolution Δz<700 nm, laser excitation 488 nm (Ar laser), 632.8 nm (HeNe laser). A PMT (photomultiplier tube) is used as the detector.

A complete solution of the active ingredient in the excipient may sometimes be difficult in producing corresponding solid compositions, so the present invention also includes such solid compositions which also contain small quantities of undissolved active ingredient particles. Such small quantities of undissolved particles do not interfere with the advantageous properties of the composition according to the invention. However, less than 15 wt %, preferably less than 10 wt %, more preferably less than 5 wt % and especially preferably less than 1 wt % of the total quantity of the active ingredient should be present in the form of particles in the solid composition. The composition according to the invention especially preferably does not contain any active ingredient particles in particular no active ingredient particles which can be observed visually, for example, under a light microscope, because of the phase boundaries occurring between the active ingredient and the excipient. The solid composition according to the invention should appear to be completely homogeneous and without any discernible phase boundaries when examined visually.

Due to the solid composition according to the invention, the active ingredient is distributed uniformly in the excipient and is thus “prediluted.” The resulting composition can be processed easily to pharmaceutical drugs either directly or with additional excipients, for example. In particular the composition according to the invention allows uniform mixing with other excipients without necessitating complex micronization. Due to the predilution of the active ingredient, furthermore, a homogeneous distribution in pharmaceutical formulations produced therefrom and thus a uniformity of the active ingredient content of these formulations are ensured. This facilitates, for example, the production of 200 mg tablets containing only 1 mg rasagiline. Another advantage of the composition according to the invention is that the molecularly dispersed distribution of the active ingredient in the excipient accelerates the dissolution of the active ingredient. This may be important, for example, when a rasagiline salt that is sparingly soluble in water or is at least less water soluble, such as rasagiline tartrate, for example, is used.

The quantity of the active ingredient in the solid composition according to the invention is not limited in particular. Instead it depends first on the desired dilution effect and secondly on the solubility of the active ingredient in the selected excipient. For example, the composition according to the invention may contain 0.5 wt % to 25 wt % active ingredient, calculated as the free base, based on the total weight of the excipient and active ingredient. In a preferred embodiment, the composition according to the invention contains 5 wt % to 15 wt % active ingredient calculated as the free base, based on the total weight of the excipient and active ingredient.

Any pharmaceutically acceptable excipient capable of forming a homogeneous, molecularly disperse mixture with rasagiline or the selected pharmaceutically acceptable salt thereof may be chosen as the excipient. The excipient must thus be capable of dissolving the active ingredient in the desired concentration. Suitable excipients include, for example, polymers, copolymers, saccharides, oligosaccharides, polysaccharides and sugar alcohols. The following excipients have proven to be particularly suitable: sucrose, sorbitol, xylitol, Eudragit, polyethylene glycol (PEG, for example, PEG 4000 or PEG 20000), polyoxyethylene glycol monostearate, glycerol polyethylene glycol ricinoleate, macrogol glycerol stearate (for example, Gelucire), glycerol palmitol stearate (for example, Precirol), macrogol glycerol laurate (for example, Gelucire 50), polyethylene glycol cetylstearyl ether (for example, Cremophor A25), glycerol monostearate (for example, Imwitor), polyvinylpyrrolidone (PVP, for example, PVP 30 or Kollidon VA64), methacrylates, cellulose derivatives such as cellulose ethers (for example, Methocel K4M CR Premium), methyl cellulose (MC), hydroxypropyl cellulose (HPC, for example, HPC HF) and hydroxypropylmethyl cellulose (HPMC, for example, HPMC 615) and copolymers like copovidone (from vinyl acetate and vinylpyrrolidone) or Pluronic, for example, Pluronic F68, a block copolymer of ethylene oxide and propylene oxide.

The composition according to the invention may contain an excipient or a mixture of two or more excipients.

The solid compositions according to the invention can be prepared by mixing the excipient and active ingredient in such a way as to yield a homogeneous, molecularly disperse mixture. For example, corresponding mixing may be performed in a combined melt of the excipient and active ingredient, preferably by melt extrusion. Alternatively, there is the option of mixing by dissolving the excipient and active ingredient in a solvent and then evaporating the solvent. In evaporating the solvent it is important to be sure that the excipient and active ingredient are not precipitated concurrently but instead form the desired homogeneous, molecularly disperse mixture. This evaporation by spray drying is less suitable, for example, because excipient particles on which the active ingredient is adsorbed in amorphous form may then be formed, i.e., in a separate phase. To prevent separate precipitation of the excipient and active ingredient from the solution, the evaporation step may be performed slowly over a longer period of time of at least 24 hours for example, preferably over a period of 24 hours to 150 hours, for example, in particular over a period of 72 hours to 120 hours.

Any solvent capable of dissolving both the active ingredient and the excipient may be used as the solvent. For example, water or a mixture of water and ethanol, for example, an approx. 20 vol % to 30 vol % aqueous ethanol solution is suitable. In addition the solution may be acidified, for example, with an organic or inorganic acid such as hydrochloric acid, acetic acid, formic acid, benzoic acid, citric acid, malic acid, tartaric acid, oxalic acid, fumaric acid, succinic acid, maleic acid and salicylic acid. Hydrochloric acid and citric acid in particular citric acid are preferred acids.

Alternatively, there is the possibility of spaying the solution onto inert excipient particles, so-called nonpareils, for example. The spraying may be performed in a fluidized bed granulator, for example. By spraying the solution onto the excipient particles, the excipient and active ingredient are precipitated from the solution jointly as a homogeneous, molecularly dispersed mixture.

The solid composition according to the invention may be processed further according to conventional methods with which those skilled in the art are familiar to form a pharmaceutical drug, in particular a solid dosage form. This is preferably a capsule, tablet, an orally disintegrating tablet, a delayed-release tablet, pellets or granules. Tablets which are produced by direct pressing with the excipients generally used for this purpose are preferred.

The pharmaceutical drug containing the solid composition according to the invention is in particular in the form of a tablet containing 0.2 wt % to 20 wt % active ingredient, 40 wt % to 95 wt % of one or more fillers, 0 wt % to 30 wt % of one or more disintegrants and 0 wt % to 5 wt % of one or more lubricants, each based on the total weight of the pharmaceutical drug without any coatings that might be present. Corresponding pharmaceutical drugs have an excellent uniformity of content which usually cannot be achieved in particular in tablets with direct pressing, in particular at a low active ingredient content of <5 wt %.

The present invention will now be explained in greater detail by the following examples without directly limiting it to these examples.

### EXAMPLE 1

Rasagiline tartrate, based on 10 g of the free base, was mixed with 100 g of a polymer or sugar alcohol in a Petri dish and heated in a heating oven at 150° C. until it melted (approx. 1 hour). The products were analyzed visually, i.e., by light microscopy and with the naked eye (for homogeneity) and by HPLC (active ingredient content and impurities). Sorbitol, PEG 4000, glycerol palmitol stearate (Precirol), macrogol glycerol laurate (Gelucire 50), PEG cetylstearyl ether (Cremophor A25) and glyceryl monostearate (Imwitor) have proven to be especially suitable solvents. The melts prepared with these excipients have also proven to be stable (visually and by HPLC) after 4 weeks of storage at 40° C. and 75% atmospheric humidity.

### EXAMPLE 2

Rasagiline tartrate based on 50 g of the free base was mixed with 500 g PEG in a free-fall mixer (Turbula TB10) for 15 minutes and extruded in a twin screw extruder (Leistritz Micro 18), whereupon multiple heatable cylinders were adjusted individually to rising temperatures from 20° C. to 65° C. along the screws. The resulting extrudate was cooled to room temperature and ground to form particle with an average size of 800 to 1000 μm.

### EXAMPLE 3

Rasagiline tartrate, based on 100 g of the free base, was mixed with 500 g Gelucire in a free-fall mixer (Turbula TB10) for 15 minutes and extruded in a twin screw extruder (Leistritz Micro 18) for 15 minutes and extruded in a twin screw extruder (Leistritz Micro 18), whereupon multiple heatable cylinders were adjusted individually to rising temperatures from 25° C. to 100° C. along the screws.

### EXAMPLE 4

The excipient and rasagiline (as the tartrate) were mixed in a weight ratio of 10:1 in a Petri dish or in a glass beaker. Purified water was added to the mixture of solids, and the entire mixture was then stirred on a magnetic stirrer until the solids were completely dissolved (approx. 2 hours). Next the solution was dried in a vacuum oven at 30° C. and 0.1 bar for 72 hours. The products were inspected visually, i.e., by light microscopy and with the naked eye (for homogeneity) and analyzed by HPLC (active ingredient content and impurities). Sorbitol provide to be an especially suitable excipient here. The solid solutions prepared with sorbitol have proven to be stable (visually and by HPLC) after 4 weeks of storage at 40° C. and 75% atmospheric humidity.

### EXAMPLE 5

The excipient and rasagiline (as the tartrate) were mixed in a weight ratio of 10:1 and/or 2:1 in a Petri dish or in a glass beaker. A 30% (volume percent) aqueous ethanol solution was added to the mixture of solids, and the entire mixture was then stirred on a magnetic stirrer until the solids were completely dissolved (approx. 2 hours). Next the solution was dried in a vacuum oven at 40° C. and 0.1 bar for 120 hours. The products were inspected visually, i.e., by light microscopy and with the naked eye (for homogeneity) and analyzed by HPLC (active ingredient content and impurities). Especially suitable excipients have proven to be PVP 30, HPMC 615 and Kollidon VA 64 in a weight ratio of 2:1, PEG 20000, HPC HF and Pluronic F68 in a weight ratio of 10:1 as well as Methocel K4M CR Premium and PEG 4000 in both weight ratios. The solid solutions prepared using these excipients in the respective weight ratios have also proven to be stable (visually and by HPLC) after 4 weeks of storage at 40° C. and 75% atmospheric humidity.

### EXAMPLE 6

The excipient and rasagiline (as the tartrate) were mixed in a weight ratio of 10:1 and/or 2:1 in a Petri dish or in a glass beaker. A 30% (volume percent) aqueous ethanol solution was added to the mixture of solids, acidified with 0.1 mol/liter HCl (0.5 mL to 100 mL solvent) and the entire mixture was then stirred on a magnetic stirrer until the solids were completely dissolved (approx. 2 hours). Next the solution was dried in a vacuum oven at 40° C. and 0.1 bar for 120 hours. The products were inspected visually, i.e., by light microscopy and with the naked eye (for homogeneity) and analyzed by HPLC (active ingredient content and impurities). Especially suitable excipients have proven to be sorbitol, PVP 30, HPMC 615, PEG 4000, PEG 20000, HPC HF and Pluronic F68, regardless of the weight ratio. The solid solutions prepared using these excipients in the respective weight ratios have also proven to be stable (visually and by HPLC) after 4 weeks of storage at 40° C. and 75% atmospheric humidity.

### EXAMPLE 7

Rasagiline tartrate, based on 10 g of the free base together with 20 g sorbitol was mixed with 200 mL of a 30% (vol %) aqueous ethanol solution. After adding 1 mL of a 0.1 molar hydrochloric acid solution, the mixture was stirred for 3 hours on the magnetic stirrer. The resulting clear solution was sprayed onto 300 g nonpareils with a diameter of 500 μm in a fluidized bed granulator (Glatt GPCG 3.1, inlet temperature 70° C., outlet temperature 45° C., spray pressure 1.6 bar, spray rate approx. 1.5 g/min).

### EXAMPLE 8

Rasagiline tartrate, based on 5 g of the free base, together with 50 g HPMC was mixed with 500 mL of a 20% (vol %) aqueous ethanol, solution. After adding 1 mL of a 0.1 molar hydrochloric acid solution, the mixture was stirred for 3 hours on the magnetic stirrer. The resulting clear solution was sprayed onto nonpareils with a diameter of 400 to 500 μm in a fluidized bed granulator (Glatt GPCG 3.1, inlet temperature 70° C., outlet temperature 45° C., spray pressure 1.6 bar, spray rate approx. 1.5 g/min).

### EXAMPLE 9

Rasagiline tartrate, based on 1 g of the free base, together with 2 g Pluronic F68 was mixed with 100 mL water and stirred for 3 hours on the magnetic stirrer. The resulting clear solution was sprayed onto nonpareils with a diameter of 500 to 600 μm in a fluidized bed granulator (Glatt GPCG 3.1, inlet temperature 70° C., outlet temperature 45° C., spray pressure 1.6 bar, spray rate approx. 1.5 g/min).

### EXAMPLE 10

An extrudate of 72.05 g rasagiline tartrate and 500 g PEG 400 according to Example 2 was prepared with the following temperature settings on the extruder: cylinder 1: 55° C., cylinder 2: 60° C., cylinder 3: 63° C., cylinder 4: 60° C., cylinder 5: 55° C., outlet nozzle 55° C., product temperature 55° C. Samples of the extrudate were welded into sealed HDPE bottles in aluminum bags and stored at 40° C. and 75% atmospheric humidity. Before storage as well as after 4, 8 and 12 weeks of storage time, samples (n=3) were analyzed. In addition to the visual assessment, the water content was determined according to Karl Fischer (KF) and the rasagiline content and the amount of impurities were determined by HPLC.

| | t₀ | 4 weeks | 8 weeks | 12 weeks |
|---|---|---|---|---|
| Appearance | slightly gray | slightly gray | slightly gray | slightly gray |
| | agglomerates | agglomerates | agglomerates | agglomerates |
| Water content | 0.4 | 0.5 | 0.4 | 0.7 |
| according | | | | |
| to KF (%) | | | | |
| Active | | | | |
| ingredient | | | | |
| content | | | | |
| according to | | | | |
| HPLC | | | | |
| Rasagiline | 80.16 | 81.13 | 79.8 | 80.1 |
| (mg/g) based | | 101.21 | 99.55 | 99.93 |
| on starting | | | | |
| value (%) | | | | |
| Impurities | | | | |
| according to | | | | |
| HPLC | | | | |
| 1-Aminoindan | 0.06 | 0.09 | 0.09 | 0.09 |
| (%) | | | | |
| Total (%) | 0.06 | 0.09 | 0.09 | 0.09 |

### EXAMPLE 11

Tablets of the following composition were prepared using the extrudate from Example 10:

| | Content/tablet | Initial weight |
|---|---|---|
| | (mg) | (g) |
| Extrudate | 11.43 | 1.37 |
| Mannitol | 150 | 18 |
| Aerosil | 1.2 | 0.14 |
| Cornstarch | 20 | 2.4 |
| Pregelatinized cornstarch | 20 | 2.4 |
| Stearic acid | 4 | 0.48 |
| Talc | 4 | 0.48 |

Mannitol, cornstarch and pregelatinized cornstarch were weighed into a glass vessel, mixed for 20 minutes with a T10B agitator mixer at 23 rpm and then screened through a screen with 500 μm mesh. Stearic acid, talc and Aerosil were screened through a screen with 250 μm mesh and mixed together with the other excipients for 5 minutes. After adding the extrudate the entire batch was mixed for another 7 minutes. Tablet were pressed from this mixture using a hand press and a pressing force of approx. 4 kN to 8 kN.

The resulting tablets were welded into sealed HDPE bottles in aluminum bags and stored at 25° C. and 60% relative humidity. Before storage and after 3 months of storage, samples (n=2) were analyzed. In addition to the visual assessment, the water content was determined according to Karl Fischer (KF) and the rasagiline content and the amount of impurities were determined by HPLC.

| | t₀ | 3 months |
|---|---|---|
| Appearance | white tablets | white tablets |
| Water content according to | 1.3 | 1.3 |
| KF (%) | | |
| Active ingredient content | | |
| according to HPLC | | |
| Rasagiline (mg/g) | 0.71 | 0.74 |
| based on initial value (%) | | 104.23 |
| Impurities according to HPLC | | |
| 1-Aminoindan (%) | below the limit of detection | 0.06 |
| Impurity 1 (%) | 0.11 | 0.12 |
| Impurity 2 (%) | 0.11 | 0.08 |
| Total (%) | 0.22 | 0.26 |

### EXAMPLE 12

Tablets of the following composition were prepared using the extrudate from Example 10:

| | Content/tablet | Initial weight |
|---|---|---|
| | (mg) | (g) |
| Extrudate | 11.43 | 1.37 |
| Avicel PH 101 | 174 | 20.88 |
| Aerosil | 1.2 | 0.14 |
| Pregelatinized cornstarch | 10 | 1.2 |
| Mg stearate | 2 | 0.24 |
| Citric acid | 2 | 0.24 |

Avicel and pregelatinized cornstarch were weighed into a glass vessel, mixed for 20 minutes at 23 rpm using a Turbula T10B agitating mixer and then screen through a screen with 500 μm mesh. Mg stearate, citric acid and Aerosil were screened through a screen with 250 μm mesh and were mixed together with the other excipients for 5 minutes. After adding the extrudate, the entire batch was mixed for 7 minutes more. Tablets were pressed from this mixture using a hand press and a pressing force of approx. 4 kN to 8 kN.

The resulting tablets were welded into sealed HDPE bottles in aluminum bags and stored at 25° C. and 60% atmospheric humidity. Before storage and after 3 months of storage, samples (n=2) were analyzed. In addition to the visual assessment, the water content was determined according to Karl Fischer (KF) and the rasagiline content and the amount of impurities were determined by HPLC.

| | t₀ | 3 months |
|---|---|---|
| Appearance | white tablets | white tablets |
| Water content according to KF | 3.7 | 3.5 |
| (%) | | |
| Active ingredient content | | |
| according to HPLC | | |
| Rasagiline (mg/g) | 0.74 | 0.76 |
| based on initial value (%) | | 102.7 |
| Impurities according to HPLC | | |
| 1-Aminoindan (%) | below the limit of | 0.06 |
| | detection | |
| Impurity 1 (%) | not detectable | not detectable |
| Impurity 2 (%) | not detectable | not detectable |
| Total (%) | not detectable | 0.06 |

### EXAMPLE 13

Rasagiline tartrate (3 g) was placed together with 6 g PVP 30, 1.5 g citric acid and 300 mL water in a glass beaker and stirred on a magnetic stirrer until completely dissolved. The resulting clear solution was sprayed onto 200 g nonpareils with a diameter of 300 μm in a fluidized bed granulator (Innojet Ventilus, inlet temperature 40° C., outlet temperature 30° C., spray pressure 1.7 bar, spray rate approx. 1 g/min). Samples of these pellets were welded into sealed HDPE bottles in aluminum bags and stored at 30° C. and 65% atmospheric humidity. Before storage and after 12 weeks of storage time, samples (n=3) were analyzed. In addition to the visual assessment, the water content was determined according to Karl Fischer (KF) and the rasagiline content and the amount of impurities were determined by means of HPLC.

| | t₀ | 12 weeks |
|---|---|---|
| Appearance | white round pellets | white round pellets |
| Water content according to | 1.4 | 1.5 |
| KF (%) | | |
| Active ingredient content | | |
| according to HPLC | | |
| Rasagiline (mg/g) | 8.39 | 8.35 |
| based on initial value (%) | | 99.52 |
| Impurities according to HPLC | | |
| 1-Aminoindan (%) | 0.06 | 0.14 |
| Impurity 1 (%) | 0.05 | 0.06 |
| Impurity 2 (%) | not detectable | 0.11 |
| Total (%) | 0.11 | 0.31 |

### EXAMPLE 14

PEG 4000 (6 g) was placed in a glass beaker together with 200 mL water and stirred on a magnetic stirrer until completely dissolved. Then the pH was adjusted to a value between 1 and 2 by adding 8 g citric acid. Next 3 g rasagiline tartrate was added and stirring was continued until the solids were completely dissolved. The resulting clear solution was sprayed onto 300 g nonpareils with a diameter of 300 μm in a fluidized bed granulator (Innojet Ventilus, inlet temperature 42° C., outlet temperature 29° C. to 33° C., spray pressure 1.7 bar, spray rate approx. 1 g/min). Samples of these pellets were welded into sealed HDPE bottles in aluminum bags and stored at 30° C. and 65% atmospheric humidity. Before storage and after 12 weeks of storage time, samples (n=3) were analyzed. In addition to the visual assessment, the water content was determined according to Karl Fischer (KF) and the rasagiline content and the amount of impurities were determined by means of HPLC.

| | t₀ | 12 weeks |
|---|---|---|
| Appearance | white round pellets | white round pellets |
| Water content according to | 1.5 | 1.5 |
| KF (%) | | |
| Active ingredient content | | |
| according to HPLC | | |
| Rasagiline (mg/g) | 1.96 | 2.06 |
| based on initial value (%) | | 105.1 |
| Impurities according to HPLC | | |
| 1-Aminoindan (%) | below limit of | below limit of |
| | detection | detection |
| Impurity 1 (%) | 0.3 | 0.45 |
| Total (%) | 0.3 | 0.45 |

### EXAMPLE 15

Sorbitol (6 g) was placed in a glass beaker together with 200 mL water and stirred on a magnetic stirrer until completely dissolved. Then the pH was adjusted to a value between 1 and 2 by adding 8 g citric acid. Next 3 g rasagiline tartrate was added and stirring was continued until the solids were completely dissolved. The resulting clear solution was sprayed onto 300 g nonpareils with a diameter of 300 μm in a fluidized bed granulator (Innojet Ventilus, inlet temperature 63° C. to 70° C., outlet temperature 34° C. to 50° C., spray pressure 2 bar, spray rate approx. 1 g/min). Samples of these pellets were welded into sealed HDPE bottles in aluminum bags and stored at 30° C. and 65% atmospheric humidity. Before storage and after 12 weeks of storage time, samples (n=3) were analyzed. In addition to the visual assessment, the water content was determined according to Karl Fischer (KF) and the rasagiline content and the amount of impurities were determined by means of HPLC.

| | t₀ | 12 weeks |
|---|---|---|
| Appearance | partially | partially |
| | agglomerated | agglomerated |
| | white round pellets | white round pellets |
| Water content according to | 1.5 | 1.5 |
| KF (%) | | |
| Active ingredient content | | |
| according to HPLC | | |
| Rasagiline (mg/g) | 3.3 | 3.34 |
| based on initial value (%) | | 101.21 |
| Impurities according to HPLC | | |
| 1-Aminoindan (%) | below limit of | below limit of |
| | detection | detection |
| Impurity 1 (%) | 0.18 | 0.23 |
| Impurity 2 (%) | not detectable | 0.06 |
| Total (%) | 0.18 | 0.29 |

### EXAMPLE 16

Tablets of the following composition were prepared using the pellets from Example 13:

| | Content/tablet | Initial weight |
|---|---|---|
| | (mg) | (g) |
| PVP pellets | 72.8 | 7.28 |
| Avicel PH 200 | 113.2 | 11.32 |
| Macrogol 4000 powder | 13.8 | 1.38 |
| Kollidon CL | 10 | 1.4 |
| PRUV | 0.2 | 0.02 |

Avicel, Macrogol and Kollidon CL were passed through a 600 μm screen and placed in a glass vessel. PRUV was screened through a 300 μm screen and added to the mixture. The weighed pellets were added and the total mixture was mixed using a free-fall mixer (Turbula T10B) for 15 minutes. The finished mixture was pressed using an eccentric press to form tablets weighing 210 mg each.

### EXAMPLE 17

Using the pellets from Example 13, tablets of the following composition were prepared:

| | Content/tablet | Initial weight |
|---|---|---|
| | (mg) | (g) |
| PVP pellets | 72.8 | 7.28 |
| Avicel PH 102 | 93.2 | 9.32 |
| Avicel PH 101 | 40 | 4 |
| Ac-Di-Sol | 3.7 | 0.37 |
| PRUV | 0.3 | 0.03 |

Avicel and Ac-Di-Sol were passed through a 600 μm hand screen and placed in a glass vessel. PRUV was screened through a 300 μm screen and added to the mixture. The weighed pellets were added and the total mixture was mixed for 15 minutes on a free-fall mixer (Turbula T10B). Next the mixture was pressed on an eccentric press to yield tablets weighing 210 mg.

1. A solid composition containing at least one pharmaceutically acceptable excipient and rasagiline or a pharmaceutically acceptable salt thereof as the active ingredient, characterized in that the excipient and the active ingredient are present in a homogeneous, molecularly disperse mixture.

2. The solid composition according to claim 1, wherein less than 15 wt %, preferably less than 5 wt % of the active ingredient, based on its total amount, is present in the form of particles.

3. The solid composition according to claim 1, containing 0.5 wt % to 20 wt % active ingredient, calculated as the free base, based on the total weight of the excipient and active ingredient.

4. The solid composition according to claim 1, wherein the pharmaceutically acceptable excipient is selected from the group consisting of polymers, copolymers, saccharides, oligosaccharides, polysaccharides and sugar alcohols.

5. The solid composition according to claim 1, wherein the pharmaceutically acceptable excipient is selected from the group consisting of sucrose, sorbitol, xylitol, Eudragit, polyethylene glycol, polyoxyethylene glycol monostearate, glycerol polyethylene glycol ricinoleate, macrogol glycerol stearate, glycerol palmitol stearate, macrogol glycerol laurate, polyethylene glycol cetylstearyl ether, glycerol monostearate, polyvinylpyrrolidone methacrylates, cellulose derivatives and copovidone.

6. A method for producing a solid composition according to claim 1, comprising the mixing of excipient and active ingredient to form a homogeneous, molecularly disperse mixture.

7. The method according to claim 6, wherein the mixing is performed in a combined melt of excipient and active ingredient.

8. The method according to claim 7, wherein the mixing is performed by melt extrusion.

9. The method according to claim 6, wherein the mixing is performed by dissolving the excipient an active ingredient in a solvent and then evaporating the solvent.

10. A solid composition obtainable by a method according to claim 6.

11. A pharmaceutical drug containing a solid composition according to claim 1.

12. The pharmaceutical drug according to claim 11, wherein it is a capsule, a tablet, an orally disintegrating tablet, a delayed-release tablet, pellets or granules.
